(19) United States
(12) Patent Application Publication
(10) Pub. No.: US 2024/0269226 A1
(43) Pub. Date: Aug. 15, 2024

(54) COMBINATION THERAPY WITH SEMAX AND CANNABIDIOL

(71) Applicant: Hallstat Capital LLC, Sheridan, WY (US)

(72) Inventor: Bruice T. Huici, Miami, FL (US)

(21) Appl. No.: 18/434,973

(22) Filed: Feb. 7, 2024

Related U.S. Application Data
(60) Provisional application No. 63/483,604, filed on Feb. 7, 2023.

(51) Int. Cl. A61K 38/08; A61K 31/00

(52) U.S. Cl. CPC A61K 38/08; A61K 31/658

(57) ABSTRACT

The invention describes novel methods of treating patients with a combination of a Semax compound and cannabinoid and compositions containing the same. For example, in an aspect the combination can be used to reduce the likelihood of cell death after ischemic stroke and aid in cell repair in damaged cells effected by ischemic stroke.

### BACKGROUND OF THE INVENTION

Semax (methionyl-histidyl-glutamyl-glycyl-phenylalanyl-prolyl-proline) also known at Glemax is approved in Russia to treat circulatory disorders, such as strokes, heart attacks, and high blood pressure. Semax is a hexapeptide that is an analogue of Adrenocorticotropic hormone (ACTH) fragment 4-10 but is devoid of hormonal activity. Semax is available in a 0.1% Solution, which is typically used for nasal drops. Semax also comes in a powder that is mixed with water to form an injectable solution. Semax also appear to be known in it's N-acetyl from (N-acetyl-methionyl-histidyl-glutamyl-glycyl-phenylalanyl-prolyl-proline). Semax has not approved by the US FDA.

Cannabidiol (CBD) is a well-known phytocannabinoid derived from the Cannabis species. It has been approved as Epidiolex® by the US FDA (Epidyolex in Europe)(100% pure cannabidiol currently available in at least a 100 mg/mL oral solution) to treat seizures (e.g., those associated with Lennox-Gastaut syndrome, Dravet syndrome, or tuberous sclerosis complex). CBD is also available in numerous cosmetic formulations with claims to various beneficial (though typically unproven) effects.

There currently no approved drugs in the US to improve ischemic stroke deficits. The only two types of drugs that are used in treatment of ischemic stroke are TPA (tissue plasminogen activator) and anticoagulants after the stroke. TPA is used to break up the clot and eliminate the stroke (or eliminate its effects as much as possible), but TPA must be used within the first four hours from the time stroke symptoms began. Outside of the initial four-hour window, the chances of having a brain bleed (hemorrhagic stroke) with TPA increases and the patient is no longer a candidate for treatment. If found outside of that four-hour window, the current treatment is a maximum of 6 months of physical therapy to reduce some of the stroke deficits. Physical therapy is limited to 6 months because studies have shown that after 6 six months minimal to no progress is seen-whatever deficits the patient has after 6 months will more than likely stay with the patient. The patient is also typically put on anticoagulants (usually Coumadin or Plavix) as preventative measures to reduce the likelihood of clot formation and another stroke. But this is strictly preventative, they have no effect in recovering from the damages caused by ischemic stroke. These are the standard treatment protocols across the US for stroke. Time is the most important factor and trying to get treated with TPA within the four-hour window, but when that is out of the question, recovery solely depends on how the patient's body responds to physical therapy.

Thus, it would be beneficial to discovery a new method of treating ischemic stroke, particularly outside of the four-hour window when TPA is indicated.

### SUMMARY OF THE INVENTION

In an aspect, there are described methods of combination therapy, comprising administering:
(a) a therapeutically effective amount of Semax (methionyl histidyl-glutamyl-glycyl-phenylalanyl-prolyl-proline) or a derivative or pharmaceutically salt thereof; and,
(b) a therapeutically effective amount of cannabidiol.

In another aspect, there are described pharmaceutical compositions, comprising:
(a) a therapeutically effective amount of Semax or a derivative or pharmaceutically salt thereof; and,
(b) cannabidiol; and,
(c) and optionally a pharmaceutically acceptable carrier.

In another aspect, there are described compounds and/or compositions for use in medical therapy.

In another aspect, there are described the use of compounds for the manufacture of a medicament for the treatment of an indication described herein.

These and other aspects, which will become apparent during the following detailed description, have been achieved by the inventors' discovery that Semax and cannabidiol can be used together to reduce the likelihood of cell death after ischemic stroke and aid in cell repair in damaged cells effected by ischemic stroke.

### DETAILED DESCRIPTION OF THE INVENTION

Exemplary aspects of the invention are described herein. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

### Definitions

The examples provided in the definitions present in this application are non-inclusive unless otherwise stated. They include but are not limited to the recited examples.

When introducing elements of the present disclosure or an aspect thereof, the articles “a”, “an”, “the” and “said” are intended to mean that there are one or more of the elements. The terms “comprising”, “including” and “having” are intended to be inclusive and mean that there may be additional elements other than the listed elements.

About includes +/−10% of the numerical value.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

CBD (Cannabidiol) is also known as 2-[(1R,6R)-3-Methyl-6-prop-1-en-2-ylcyclohex-2-en-1-yl]-5-pentylbenzene-1,3-diol and has the structure:

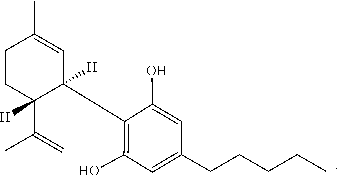 [SMILES: C=C(C)[C@@H]1CCC(C)=C[C@H]1c1c(O)cc(CCCCC)cc1O]

“Patient,” “subject” or “host” to be treated by the subject method may mean either a human or non-human mammal.

“Pharmaceutically acceptable salts” refer to derivatives of the disclosed compounds wherein the parent compound is modified by making acid or base salts thereof.

Semax (Met-Glu-His-Phe-Pro-Gly-Pro) is also known as Glemax, L-Met-L-α-Glu-L-His-L-Phe-L-Pro-Gly-L-Pro, and L-methionyl-L-α-glutamyl-L-histidyl-L-phenylalanyl-L-prolylglycyl-L-proline and has the structure:

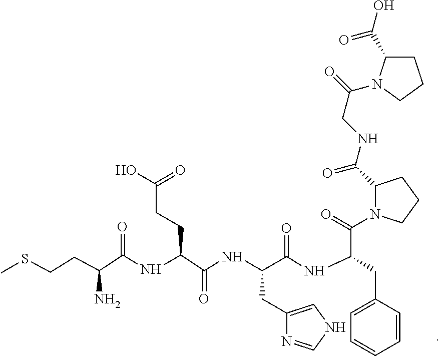

N-acetyl-Semax (N-acetyl-L-Met-L-α-Glu-L-His-L-Phe-L-Pro-Gly-L-Pro) also known as N-acetyl-Met-α-Glu-His-Phe-Pro-Gly-Pro and N-acetyl-L-methionyl-L-α-glutamyl-L-histidyl-L-phenylalanyl-L-prolylglycyl-L-proline and has the structure:

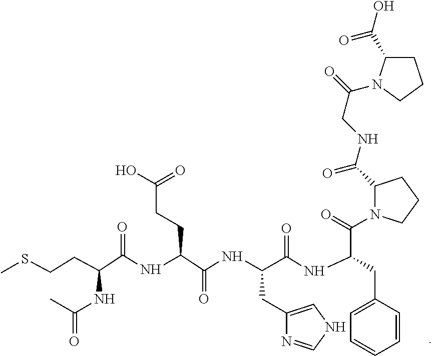

“Therapeutically effective amount,” “effective amount,” or “pharmaceutically effective amount” are used interchangeably and are defined to mean the amount or quantity of Semax and/or cannabidiol that is sufficient to elicit an appreciable biological response when administered to a patient. It will be appreciated that the precise therapeutic dose will depend on the age and condition of the patient and the nature of the condition to be treated and will be at the ultimate discretion of the attendant physician (or medical personnel). The combination of compounds can be a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

### Methods and Compositions

In an aspect, the invention involves methods of treating a patient having suffered an ischemic stroke, comprising administering to the patient having suffered an ischemic stroke:
(a) a therapeutically effective amount of Semax (methionyl histidyl-glutamyl-glycyl-phenylalanyl-prolyl-proline) or a derivative or pharmaceutically salt thereof; and,
(b) a therapeutically effective amount of CBD (cannabidiol).

In another aspect, the invention involves a method of combination therapy, comprising:
(a) administering, to a patient in need thereof, a therapeutically effective amount of Semax (methionyl histidyl-glutamyl-glycyl-phenylalanyl-prolyl-proline) or a derivative or a pharmaceutically salt thereof; and,
(b) administering a therapeutically effective amount of CBD (cannabidiol);
wherein the patient has suffered an ischemic stroke.

Ischemic stroke patients have damaged neurons due to the lack of oxygen during stroke. These patients require those neurons to regenerate to get back to normal function (or as close as possible). Ischemic stroke patients are currently prescribed an anticoagulant to reduce the chances of a second stroke. But these patients would also benefit from neuroprotection in case a second stroke occurs.

While not wishing to be bound by theory, it is expected that a benefit of treating a patient having suffered ischemic stroke with the combination of a Semax compound and CBD will be to aid in the regeneration of neurons damaged due to the ischemic stroke. Thus, in some aspects, the combination therapy is for the reduction of the likelihood of cell death after ischemic stroke and/or aid in cell repair in damaged cells effected by ischemic stroke. As a secondary benefit, it is expected that the combination therapy will also provide neuron protection (neuroprotection) in the event of a second stroke.

In some aspects, the combination therapy is for the reduction of stroke deficits outside of the four-hour window in which TPA is given. Thus, in some aspects, the combination there is given to a patient having suffered an ischemic stroke at least about 4 hours prior to initiation of the combination therapy. Other examples include starting the therapy at least about 5, 6, 7, 8, 9, 10, 11, 12, 13, 14, 15, 16, 17, 18, 19, 20, 21, 22, 23, 24, 48, 72, 96, or more hours after the ischemic stroke occurred.

In some aspects, the patient needs neuroprotection. Patients needing neuroprotection are typically patients who have previously suffered an ischemic stroke. Another set of patients would be those who are at high risk for a first ischemic stroke.

In some aspects, the combination therapy is for an indication selected from Parkinson's disease, autism, and Alzheimer's disease.

In some aspects, the combination therapy is for an indication selected from protect the cardiovascular system, counteract stress, stimulating neurogenesis, treating cognitive degeneration, treating oxidation, and treating hypoxia.

In another aspect, the invention involves a combination pharmaceutical composition, comprising:
(a) a therapeutically effective amount of Semax (methionyl histidyl-glutamyl-glycyl-phenylalanyl-prolyl-proline) or a derivative or a pharmaceutically salt thereof;
(b) a therapeutically effective amount of CBD (cannabidiol); and,
(c) optionally, a pharmaceutical acceptable carrier.

In some aspects, the pharmaceutical composition, comprises: a pharmaceutical acceptable carrier.

In some aspects, the pharmaceutical composition is in liquid form. In some aspects, the liquid form is suitable for at least one route of administration selected from nasal (e.g., nasal drops), sublingual (e.g., sublingual drops), subcutaneous, and intravenous.

In some aspects, the pharmaceutical composition is in a solid form for oral administration. In some aspects, the solid form is suitable for at least one route of administration selected from sublingual (e.g., a sublingual pill or film) and buccal. Examples of solid forms include tablets, film, troches, capsules, and caplets and gel caps.

In some aspects, the purity of the CBD is at least about 85, 86, 87, 88, 89, 90, 91, 92, 93, 94, 59, 96, 97, 98, 99, 99.5, to/and 100% pure. Purity refers to the weight % of cannabidiol as compared to other cannabis-related chemicals (e.g., a form of THC (tetrahydrocannabinol)).

In some aspects, from about 0.5-20 mg of the Semax compound is administered. Examples include about 0.5, 0.6, 0.7, 0.8, 0.9, 1, 2, 3, 4, 5, 6, 7, 8, 9, 10, 11, 12, 13, 14, 15, 16, 17, 18, 19, to/and 20 mg.

In some aspects, from about 1 mg/kg-5 mg/kg of CBD is administered. Examples include about 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, to/and 5 mg/kg.

In some aspects, from about 50-400 mg of CBD is administered. Examples include about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, to/and 400 mg.

In another aspect, the invention involves a method of combination therapy, comprising:
(a) administering, to a patient in need thereof, about 0.5-20 mg of Semax (methionyl histidyl-glutamyl-glycyl-phenylalanyl-prolyl-proline) or a derivative or a pharmaceutically salt thereof; and,
(b) administering, about 1-5 mg/kg of CBD (cannabidiol).

In another aspect, the invention involves a method of combination therapy, comprising:
(a) administering, to a patient in need thereof, about 0.5-20 mg of Semax (methionyl histidyl-glutamyl-glycyl-phenylalanyl-prolyl-proline) or a derivative or a pharmaceutically salt thereof; and,
(b) administering, about 100-300 mg of CBD (cannabidiol).

In another aspect, the invention involves a method of combination therapy, comprising:
(a) administering, to a patient in need thereof, about 10-14 mg of Semax (methionyl histidyl-glutamyl-glycyl-phenylalanyl-prolyl-proline) or a derivative or a pharmaceutically salt thereof; and,
(b) administering, about 100-300 mg of CBD (cannabidiol).

In another aspect, the invention involves a method of combination therapy, comprising:
(a) administering, to a patient in need thereof, about 12 mg of Semax (methionyl histidyl-glutamyl-glycyl-phenylalanyl-prolyl-proline) or a derivative or a pharmaceutically salt thereof; and,
(b) administering, about 150 mg of CBD (cannabidiol).

In some aspects, the pharmaceutical composition is a liquid pharmaceutical composition, comprising about 0.01-2 weight % of the Semax compound. Examples include about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, to/and 2 weight %.

In some aspects, the pharmaceutical composition is a liquid pharmaceutical composition, comprising about 0.1-20 weight % of CBD. Examples include about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to/and 20 weight %.

In another aspect, the invention involves liquid pharmaceutical compositions, comprising:
(a) about 0.01-2 weight % of Semax or a derivative or pharmaceutically salt thereof; and,
(b) about 0.1-20 weight % of cannabidiol; and,
(c) and a pharmaceutically acceptable carrier.

In another aspect, the invention involves liquid pharmaceutical compositions, comprising:
(a) about 0.05-0.2 weight % of Semax or a derivative or pharmaceutically salt thereof; and,
(b) about 0.625-2.5 weight % of cannabidiol; and,
(c) and a pharmaceutically acceptable carrier.

In another aspect, the invention involves liquid pharmaceutical compositions, comprising:
(a) about 0.1 weight % of Semax or a derivative or pharmaceutically salt thereof; and,
(b) about 1.25 weight % of cannabidiol; and,
(c) and a pharmaceutically acceptable carrier.

Semax having two (2) carboxylic acids (from the glutamine and terminal proline) can form carboxylic acid salts (e.g., a CO₂⁻Na⁺ salt (sodium or Na salt)). Semax having a primary amine (from the terminal methonine) can also form an ammonium salt (e.g., a NH₃⁺Cl⁻ salt).

N-acetyl Semax having two (2) carboxylic acids (from the Glutamine and terminal Proline) can form carboxylic acid salts (e.g., a CO₂⁻Na⁺ salt (sodium or Na salt)). The primary amine in Semax forms an amide in N-acetyl Semax.

In some aspects, the Semax compound is Semax itself.

In some aspects, the Semax compound is a pharmaceutically acceptable salt of Semax.

In some aspects, the Semax compound is sodium salt of Semax.

In some aspects, the Semax compound is an ammonium chloride salt of Semax.

In some aspects, the Semax compound is N-acetyl-Semax.

In some aspects, the Semax compound is a pharmaceutically acceptable salt of N-acetyl Semax.

In some aspects, the Semax compound is a sodium salt of N-acetyl-Semax.

In some aspects, the Semax compound is an ammonium chloride salt of N-acetyl Semax.

In some aspects, the combination of the Semax compound and CBD is once during a 24 hour period (e.g., once daily or QED).

In some aspects, the combination of the Semax compound and CBD is administered 2 times during a 24 hour period (e.g., twice daily or QED). In these aspects, the dose of Semax and CBD is typically half the single, daily dose. So, two of these doses equate to a single, daily dose.

In some aspects, the combination of the Semax compound and CBD is administered more than 2 times during a 24 hour period. Further examples include 3, 4, 5, and 6 times during a 24 hour period.

In another aspect, the invention involves compounds and/or compositions for use in medical therapy.

In another aspect, the invention involves the use of compounds for the manufacture of a medicament for the treatment of an indication described herein.

In another aspect, the invention involves a medicament, comprising:
(a) a therapeutically effective amount of Semax (methionyl histidyl-glutamyl-glycyl-phenylalanyl-prolyl-proline) or a derivative or a pharmaceutically salt thereof;
(b) a therapeutically effective amount of CBD (cannabidiol); and,
(c) optionally, a pharmaceutical acceptable carrier.

In some aspects, the medicament, comprises: a pharmaceutical acceptable carrier.

In another aspect, the method of treatment is applied to a mammal. In another aspect, the mammal is a human being.

The invention may be embodied in other specific forms without departing from the spirit or essential attributes thereof. This invention encompasses all combinations of aspects of the invention noted herein. It is understood that any and all aspects of the invention may be taken in conjunction with any other aspect or aspects to describe additional embodiments. It is also to be understood that each individual element of the aspects is intended to be taken individually as its own independent aspect. Furthermore, any element of an embodiment is meant to be combined with any and all other elements from any aspect to describe an additional aspect.

### Formulation and Dosage

Formulation of pharmaceutical compositions in tablets, capsules, and topical gels, topical creams or suppositories is well known in the art and is described, for example, in United States Patent Application Publication No. 2004/0023290 by Griffin et al. Formulation of pharmaceutical compositions as patches such as transdermal patches is well known in the art and is described, for example, in U.S. Pat. No. 7,728,042 to Eros et al. Lyophilized dosage fills are also well known in the art.

When the pharmacologically active compound in a pharmaceutical composition possesses a sufficiently acidic, a sufficiently basic, or both a sufficiently acidic and a sufficiently basic functional group, these group or groups can accordingly react with any of several inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, .beta.-hydroxybutyrates, glycolates, tartrates, methane-sulfonates, propanesulfonates, naphthalene-1-sulfonates, naphthalene-2-sulfonates, and mandelates.

If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The amount of a given pharmacologically active agent in a unit dose of a pharmaceutical composition will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating, and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the complex being used, the composition formulated, the mode of administration and the site, host, disease, and/or condition being treated. Actual dosage levels of the active ingredients in the pharmaceutical compositions of the invention can be varied to obtain an amount of the active ingredient which is effective to achieve the desired therapeutic response for a particular subject, composition, and mode of administration, without being toxic to the subject. The selected dosage level depends upon a variety of pharmacokinetic factors including the activity of the therapeutic agent, the route of administration, the time of administration, the rate of excretion of the compound being employed, the severity of the condition, other health considerations affecting the subject, and the status of liver and kidney function of the subject. It also depends on the duration of the treatment, other drugs, compounds and/or materials used in combination with the therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20.sup.th ed., 2000. Optimal dosages for a given set of conditions can be ascertained by those skilled in the art using conventional dosage-determination tests in view of the experimental data for an agent.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, e.g., in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-.alpha.-naphthylamine, or lecithin can be used. Also, chelators such as EDTA can be used.

Other ingredients that are conventional in pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the invention is contemplated. Supplementary active ingredients can also be incorporated into the compositions, particularly as described above.

For administration intranasally or by inhalation, the compounds for use according to the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days; in other alternatives, depending on the therapeutic agent and the formulation employed, release may occur over hours, days, weeks, or months. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Pharmaceutical compositions according to the invention can be prepared in accordance with methods well known and routinely practiced in the art. See, e.g., Remington: The Science and Practice of Pharmacy, Mack Publishing Co., 20.sup.th ed., 2000; and Sustained and Controlled Release Drug Delivery Systems, J. R. Robinson, ed., Marcel Dekker, Inc., New York, 1978. Pharmaceutical compositions are typically manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

All references listed herein are individually incorporated herein in their entirety by reference.

Numerous modifications and variations of the invention are possible considering the above teachings. It is therefore to be understood that within the scope of the appended claims, the invention may be practiced otherwise than as specifically described herein.

1. A method of combination therapy, comprising:
(a) administering, to a patient in need thereof, a therapeutically effective amount of Semax (methionyl histidyl-glutamyl-glycyl-phenylalanyl-prolyl-proline) or a derivative or a pharmaceutically salt thereof; and,
(b) administering a therapeutically effective amount of CBD (cannabidiol);
wherein the patient has suffered an ischemic stroke.

2. The method of claim 1, wherein the combination therapy, comprising:
(a) administering, to a patient in need thereof, about 0.5-20 mg of Semax or a derivative or a pharmaceutically salt thereof; and,
(b) administering, about 1-5 mg/kg of CBD (cannabidiol).

3. The method of claim 1, wherein the combination therapy, comprising:
(a) administering, to a patient in need thereof, about 0.5-20 mg of Semax or a derivative or a pharmaceutically salt thereof; and,
(b) administering, about 100-300 mg of CBD (cannabidiol).

4. The method of claim 1, wherein the combination therapy, comprising:
(a) administering, to a patient in need thereof, about 10-14 mg of Semax or a derivative or a pharmaceutically salt thereof; and,
(b) administering, about 100-300 mg of CBD (cannabidiol).

5. The method of claim 1, wherein the combination therapy, comprising:
(a) administering, to a patient in need thereof, about 12 mg of Semax or a derivative or a pharmaceutically salt thereof; and,
(b) administering, about 150 mg of CBD (cannabidiol).

6. The method of claim 1, wherein the Semax compound is selected from:
a. Semax;
b. a pharmaceutically acceptable salt of Semax;
c. a sodium salt of Semax;
d. an ammonium chloride salt of Semax;
e. N-acetyl-Semax;
f. a pharmaceutically acceptable salt of N-acetyl Semax;
g. a sodium salt of N-acetyl-Semax; and,
h. an ammonium chloride salt of N-acetyl-Semax.

7. The method of claim 1, wherein the Semax compound is Semax.

8. The method of claim 1, wherein the Semax compound is a pharmaceutically acceptable salt of Semax.

9. The method of claim 1, wherein the Semax compound is a sodium salt of Semax.

10. The method of claim 1, wherein the Semax compound is an ammonium chloride salt of Semax.

11. The method of claim 1, wherein the Semax compound is a pharmaceutically acceptable salt of N-acetyl-Semax.

12. The method of claim 1, wherein the Semax compound is a sodium salt of N-acetyl-Semax.

13. The method of claim 1, wherein the Semax compound is an ammonium chloride salt of N-acetyl-Semax.

14. The method of claim 1, wherein the therapy is initiated at least about 4 hours after the patient suffered the ischemic stroke.

15. A pharmaceutical composition, comprising:
(a) a therapeutically effective amount of Semax or a derivative or a pharmaceutically salt thereof;
(b) a therapeutically effective amount of CBD (cannabidiol); and,
(c) optionally, a pharmaceutical acceptable carrier.

16. The pharmaceutical composition of claim 15, comprising: a pharmaceutical acceptable carrier.

17. The pharmaceutical composition of claim 15, wherein the pharmaceutical composition is in liquid form.

18. The pharmaceutical composition of claim 15, wherein the pharmaceutical composition is in solid form.

19. The pharmaceutical composition of claim 15, wherein the Semax compound is selected from:
a. Semax;
b. a pharmaceutically acceptable salt of Semax;
c. a sodium salt of Semax;
d. an ammonium chloride salt of Semax;
e. N-acetyl-Semax;
f. a pharmaceutically acceptable salt of N-acetyl Semax;
g. a sodium salt of N-acetyl-Semax; and,
h. an ammonium chloride salt of N-acetyl-Semax.

20. The pharmaceutical composition of claim 15, wherein the Semax compound is Semax itself.
